(19) United States
(12) Patent Application Publication
(10) Pub. No.: US 2021/0290871 A1
(43) Pub. Date: Sep. 23, 2021

(54) AUTOMATIC AEROSEL AND DROPLET HYBRID SANTIZATION SYSTEM FOR DENTISTRY

(71) Applicant: Dentsafe Biomedical Incorporated, Vancouver (CA)

(72) Inventors: Seyadali MOSAVI, Eshafan (IR); Vida Kargar DEHNAVI, Eshafan (IR)

(21) Appl. No.: 17/169,822

(22) Filed: Feb. 8, 2021

(30) Foreign Application Priority Data
Mar. 18, 2020 (IR) 13965014000301498
Apr. 8, 2020 (IR) 13995014000300034

(51) Int. Cl. A61M 16/00; A61L 9/16

(52) U.S. Cl. CPC A61M 16/009; A61L 9/16

(57) ABSTRACT

One of the main problems of hoods and central ventilation devices for dental offices and clinics is the lack of fluid suction between the patient and the dentist during the spraying of the aerosol and droplet. This disclosure provides a solution to build a new device using technologies that follow the principles of ergonomics in designing and using the calculated discharge system to limit the movement of the aerosol and the droplet. This device proposes an isolation process whereby fluid transfer, along with the contaminated particles attached to the negative ions, are trapped in the chamber and transfer to the filter due, to prevent diffusion to the environment. This device also utilizes and control of optimal parameters of heat and light and negative ions, ultraviolet and ozone to sterilize and eliminate viruses and bacteria.

### CROSS-REFERENCE TO RELATED APPLICATION

This application relies on disclosure of and claims priority to and benefit of filing date of Islamic Republic of Iran Provisional application No. 139650140003014980, filed on Mar. 18, 2020, and Islamic Republic of Iran utility application No. 139950140003000342, filed on Apr. 8, 2020, the disclosure of which is incorporated herein by reference in its entirety.

### BACKGROUND

This disclosure relates to an isolation and sterilization system for dental personal protective equipment in biomedical engineering.

According to the production process, this disclosure is related to engineering and dentistry and according to the technical innovation and its background of art; it is related to bio mechatronics.

In dental clinics, there are two main routes of infection through fluid transfer. The first case is respiratory infection through inhalation of droplets and aerosol particles (aerosol/droplet) to the dentist and assistant's face and the second one, contact infection through droplets and aerosol particles that adhere to the environment and dentist's cloth.

### TECHNICAL PROBLEM

The use of personal protective equipment (PPE) such as dental hoods and air condition in dental clinics and offices is one of the requirements for optimal ventilation of these environments. In dental treatment and diagnostic procedures, patient breathing and air/water input produced by rotating dental equipment and high-speed ultrasonic devices cause the aerosol to spread, and this fluid compound is directed to the dentist's face and poses a threat of infection or contamination to the dentist and the dentist's assistant.

Dental hood and ventilation systems in the field of medicine and health are very important in hospitals, medical, and dental clinics, and very little research has been done in the use of volumetric cross-sectional isolation devices in this field.

Several problems are obvious in these fluid transfer systems that are some of the limitations of the systems and they are listed here: The first point: due to the use of hoods and ventilation systems centrally in clinics and hospitals that are done through related channels and lack of sterilization of these systems such as level 3 and 4 clinical laboratories, these systems have many problems with the spread and transmission of fluid contamination within the system.

High price and non-use in non-standard offices and clinics are other problems of these examples. Lack of sterility of central system transmission channels and non-sterilization of cross-section hood systems simultaneously are other problems of these devices.

### SUMMARY

Dentists are regularly challenged to pandemic because of COVID 19 processes and equipment that protect employees from occupational hazards. he air and blood borne pathogens standard to establish in greater detail the requirements that dental clinics staffs must identify and make use of appropriate, effective, and safer medical devices. In the past, protecting employees from airborne pathogens primarily involved personal protective equipment (PPE), such as gloves, masks, and protective eyewear. Although PPE is an essential component of infection control and occupational safety does and not fully protect employees against virus infection completely. Engineering isolated controls device to remove a hazard from the workplace should be develop for maximize the safety in clinics.

The transmission and spread of viral and bacterial diseases, especially SARA-CoV-2, through aerosol and droplet due to the proximity of patients and dentists and the spraying of fluid droplets containing the virus due to the use of rotary tools such as Angles and turbines have made dentistry a dangerous job. The design goals of this isolated ergonomic device are to improve working conditions and prevent the transmission of pathogens such as (SARA-CoV-2 virus) for dentists and patients. The use of this device in offices and clinics is to prevent the virus transmission in fluids from entry and exit of liquids (droplet) and air transfer (aerosol) and saliva jump and other liquid particles to the dentist.

One of the main problems of hoods and central ventilation devices for dental offices and clinics is the lack of fluid suction between the patient and the dentist during the spraying of the aerosol and droplet. This disclosure provides a solution to build a new device using technologies that follow the principles of ergonomics in designing and using the calculated discharge system to limit the movement of the aerosol and the droplet. This device proposes an isolation process whereby fluid transfer, along with the contaminated particles attached to the negative ions, are trapped in the chamber and transfer to the filter due, to prevent diffusion to the environment. This device also utilizes and control of optimal parameters of heat and light and negative ions, ultraviolet and ozone to sterilize and eliminate viruses and bacteria.

### BRIEF DESCRIPTION OF THE DRAWINGS

FIG. 1 is a schematic diagram showing a design of the whole system and horizontal, vertical and lateral view and final assembly with introduction of components.

FIG. 2 is a schematic diagram showing a design of Chamber air suction box components.

FIG. 3 is a schematic diagram showing a design PCB of Control modules.

FIG. 4 is a schematic diagram showing a design of computer application control program.

FIG. 5 is a diagram illustrating an electrical board controller for an exemplary device.

### DETAILED DESCRIPTION

### Solution to Problem

By comparing the technical knowledge and previous solutions to solve the problems, the new device with knowledge and the existing innovative solutions will provide special and practical benefits that can be extracted and understood. Unlike previous medical ventilation systems and hoods in the world, this device has the ability to locally filter, isolate, and discharge the droplet.

This device also has the sterilize ability to control infection and viral or bacterial transmission in combination by correcting the structure of the dentist's posture. Due to the possibility of placing the ultraviolet (UV) lamp, it has the ability to sterilize the inner chamber and the surface of the tab.

Using a device that combines several stages and functions such as high-speed fluid suction technology ionized with a negative ion with two stages of virus filter, air blast for better discharge in the section between patient and dentist, high heat blast to eliminate the cell wall of COVID-19 virus and sterilization of the system with ultraviolet C (UVC) light are not observed in any research or sample.

The Portable ergonomic dental isolator device to prevent the transmission of infectious diseases specially the COVID-19, which is designed using a combination of science and technology and with new innovations, considers solutions to solve the mentioned problems as targets that are mentioned below. The overall goals and innovations of this device include eliminating the way of virus transmission from patient to dentist, and vice versa, ensuring the quality, comfort, and managing the risk of disease transmission are other benefits of using this product.

### Advantageous Effects of Disclosure

With this innovative product, control of transmission disease, improvement of work efficiency and comfort of the dentist, cost reduction, and accuracy are achieved. The portability of ergonomic design for easy work and cheap product is one of the unique features of this product, which can also be used with a function to sterilize office space with UV.

Ergonomic design based on the standard distance between the patient's head and the dentist's head in different modes based on the clock technique and correcting the dentist's posture is one of the innovative goals of this device.

The device includes a suction turbo fan and a tail turbo fan is calculated simultaneously with the distance and this design has been done to better transfer the fluid for the filtration process and for better displacement of the drop in the sucked fluid path, which is one of the most important goals.

The system with a chamber has the ability to filter particles containing viruses and bacteria, and sterilization of the entire chamber at the end of working with ultraviolet light, is a health requirement and the main goals. The use of wireless control, which is remotely connected to a computer, pedal and even Android application software, is one of the features of this design.

The use of UVC lamp system to sterilize the environment is very practical, which is one of the minor goals of this device. Due to the mentioned mechanisms, this device has the ability to sterilize and cross-insulate the transmission in the field of health in clinics and hospitals.

### Detailed Description of Embodiments

Reference will now be mail in detail to various exemplary embodiments of the disclosure. It is to be understood that the following of exemplary embodiment is not reader more detailed understanding of certain aspect and features on the disclosure. Outflow of these fluids may contain dentin and enamel particles and carious tooth tissue, bacteria and viruses in the patient's mouth, water compressed by turbine and angle, along with very small droplets of blood and saliva.

Transmission of microorganisms contain pathogenic bacteria, viruses and fungi, as well as direct transmission of highly infectious diseases such as Mycobacterium tuberculosis, Hepatitis B and C, Staphylococci, Herpes simplex virus 1 and 2, Influenza, Corona, SARS and defective virus The immune system are some of the transmission risks in dentistry.

According to official WHO data, respiratory infections can be transmitted through droplets of various sizes: When the droplets are 5-10 micrometers in diameter, they are called droplets, and when they are less than 5 micrometers in diameter, they are called aerosols. According to the available evidence, COVID-19 virus is primarily transmitted to humans through respiratory droplets and contact routes. Droplet transmission occurs when a person is in close contact (at a distance of 1 meter) with a person with respiratory symptoms (such as coughing or sneezing) and even spraying these particles with dental rotating devices at a greater distance can be transmitted through the mouth, nose, or eyes

Therefore, the COVID-19 virus transmission can be through direct contact with infected people and indirect contact with air. In terms of fluid transfer, the emission of droplets is different, and the presence of viruses in droplets can be cited as the main cause of disease transmission, which these particles with a diameter of 5 μm can remain in the air for a long time and can be transmitted from others to others over long distances.

Droplets less than 10 μm in diameter are small aerosols that are carried by ventilation or wind and can therefore move around the rooms. What makes N95 face masks different from surgical masks is that the previous models were designed to prevent airborne particles: They must filter 95% of droplets smaller than 0.3 mm.

Droplets larger than 10 micrometers have the ability to spread and up to a maximum distance of 0.11 mm or more. Indeed, the droplets can be even larger, so much so that spraying droplets from coughing or sneezing (diameter 0.1 mm and above) can be seen with the naked eye. Calculations show that if exhaled droplets less than 0.1 mm may evaporate depending on size, humidity and temperature or fall within a radius of 2 meters. But coughing or sneezing can expels droplets 50 meters per second (for sneezing) or 10 meters per second (for coughing) from the mouth like projectiles and the drops can go as far as 6 meters.

In this case, the “safe distance” mentioned in the sources is generally 6 feet, which may not be sufficient in social and dental encounters, except that the dentist wears a mask. Droplets Larger than aerosol evaporate at a rate of less than 1 meter per second when exhaled or fall to a distance of less than 1.5 meters. Larger droplets (less than 0.1 mm) can be spread at high speeds through coughing or sneezing or by Angles and dental turbines more than 2 m or 10 m, respectively.

The dental clinic is a common medical center where aerosol particles (air/drops) are produced during various stages of treatment. These treatments include the following: Ultrasonic devices, air purifiers, tooth preparation with rotating tools at high and low speeds, use of water syringes and air spraying, etc. Spraying of aerosol as well as particles and liquids containing the virus during dental procedures is obvious to the dentist, dental team, and patient. According to research and published articles, air purifiers (dental hoods) are unlikely to remove droplets/aerosols, and do not have the effect of controlling definitive infections in dental clinics. The rotational speed of dental Angle and drills in the range of 30,000 rpm has produced more aerosols and the rotation speed of the dental turbines used in this clinic, which produces more droplets, is 300,000 rpm. The frequency of the ultrasonic piezoelectric vibration is 30,000 Hz, which is within the standard range.

The initial speed of droplet or aerosol spread from the patient's mouth during dental treatment can be estimated at 30 meters per second. Numerous studies have reported that dentists and patients are at high risk for infection with various types of bacteria and viruses and due to this problem, the community dental in the world has been banned treatments that cause droplets and aerosols due to the pandemic of COVID-19 virus.

The use of surgical masks and even N95 and glasses, Shield, with advanced ventilation devices can only protect 90 to 95 percent, which has many problems such as the passage of viruses through the common masks and also the problem of respiratory pressure drop, heat, and mask pressure to the face greatly reduces the ability to work. Another problem is the remaining of these spread droplets on the dentist's clothes and the surrounding environment, which requires a complete replacement and sterilization for each patient, and can sometimes be contagious due to human error and also time consuming and costly. Bacterial and viral contamination in the air has particles with positive and negative charges. From the accumulation of positively charged particles and ions, a positive ion is formed. And from the accumulation of negative charged particles and ions, a negative ion is formed. Electric discharge of high voltage through the operation of the electrode can produce a large amount of negative ions. These negative ions can adhere to the aerosol and droplet containing bacteria and viruses in the air and clean the air from contaminants.

The use of hoods and ventilation alone in research and disclosure has specific applications in sterile and ventilated health environments. Due to the main and secondary goals mentioned, the creation of a device that has the ability to isolate and sterilize to prevent the spread of disease in the field of health and dentistry is much needed and is not seen in previous disclosures and research.

By designing and modifying all these complex technologies in the form of a single platform with control, this disclosure is introduced. The disclosures that have been used as literature review in this field are introduced below.

The disclosure is related to the design and construction of a dental hood with a virtual wall. This system is sucked by a metal funnel system and work with an oxygen blower to the patient's face, freely without direct connection to the respiratory system (Iranian Patent 78853 with Declaration No. 13115114111318771).

In another disclosure, a medical gas ventilator with a system for sucking and collecting medical gases, smoke, stains and particles around the patient's face is used is disclosed in patent application WO1996006651A1.

In related embodiment the dental separation system, a system has been implemented to separate the patient and the dentist from the products of fracture of the tooth decay, including the toxic mercury vapor produced during the removal of amalgam. The system contains an extraction assembly, fiber optic lighting system, gas and water discharge method, and compressed air surface cleaning system is disclosed in patent application U.S. Pat. No. 4,917,603 A.

Other related embodiment is the U.S. patent issued on Mar. 23, 1993, shows a suction tube in a position higher than the patient's face is disclosed in patent application U.S. Pat. No. 5,195,512 A.

The U.S. patent issued on Feb. 28, 1989, reveals a device in the form of a suction mask with a modified cup geometric shape on the outside is disclosed in patent application U.S. Pat. No. 4,807,617 A.

The U.S. patent issued on Apr. 15, 1975, which reveals a hood or hollow shield mechanism, is drilled on one side and connected to a suction line is disclosed in patent application U.S. Pat. No. 3,877,837 A.

In various embodiment is used as a stream of reducing materials and performs sterilization of disposable dental equipment is disclosed in patent application US6322568B1.

Positive and negative ions move toward the third electrode while being accelerated. Then, it is sent from the outlet to the outside of the casing. The ion feeder according to the present disclosure has a And a ground facing the first electrode. Plasma driven catalyst system with negative ion disclosed for disinfection and purification of gases is disclosed in patent application US9138504B2.

Using air filter with combined enhanced collection efficiency and surface sterilization disclosed is disclosed in patent application US6245132B1.

Sterilizing method, sterilizing apparatus, and air cleaning method and apparatus using the negative ion is disclosed in is disclosed in patent application U.S. Pat. No. 8,404,182 B2.

Device for microbe and virus capture and inactivation is disclosed in is disclosed in patent application EP2578243B1.

There is no internal disclosure that conforms to the specifications of this disclosure. Provide a solution to the existing technical problem along with a detailed, sufficient, and integrated description of the disclosure.

Due to the background of existing research and disclosures in the world, each one has its own limitations, which due to the complexity of droplet spread along with bacteria and viruses such as COVID-19 and their different contagious behavior requires a comprehensive and complete system which to be able to reduce the risk of disease contagious in the field of dentistry in hospitals and clinics with a special technology in the form of a comprehensive device and structural modifications postural correction treats dental treatment according to ergonomic design.

There are four three processes involved in how this disclosure works, such as turbo suction, negative plasma ion, hot air blast, and air vent filter containing and destroying virus in droplets and aerosol, and the whole system can be sterilization with ozone and UVC and with ergonomic system design, this system will corrects the dentists or dental hygienist posture.

The use of programmed wireless control and performance of each part of the device, as well as the construction of modules in smart control with special capabilities such as heat, light, ionization, air blast and air suction simultaneously or separately are the distinctive features of this device.

### Brief Description of Drawings

Specific aspects of this disclosure consist of three separate but related but related aspect 1 and 2 and 3. Aspect 1 is the mechanical unit mechanism and the aspect 2 is electrical control system and aspect 3 is control application.

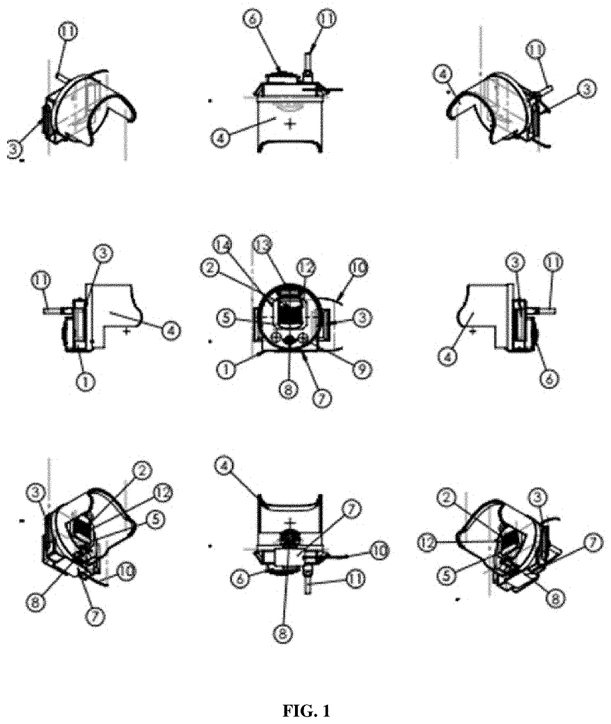

FIG. 1 is a schematic side sectional view of an ion feeder according to a first embodiment of the present disclosure.

Aspect 1 in comprising fluid transfer mechanism with fluid mechanics and electronic unit methods including lights, UVC lamps, ozone and ionization voltage systems, extra oral camera and wireless control, and the body design ergonomic mechanism.

Aspect 1 unit includes All accessories are connected to the chamber and are located on the base along with the home screen. There are control circuit, wiring, filters, transformer and suction turbo fan inside the chamber (FIG. 1 Part 1).

According to aspect 1, the primary plate filter, containing a disposable Nano fiber layer, is located under the entrance lattice of the chamber door. The Turbo dual suction fan is used to suck the droplet and aerosol spread from the patient's mouth and transfer them to the filtering system under this filter (FIG. 1 Part 2).

According to aspect 1, two UVC lamps are housed inside a rectangular chamber with a 160 degree angle on either side. The box with the UVC lamp on either side of the chamber is located at a relatively suitable angle of the chamber to avoid contact of liquid with lights (FIG. 1 Part 3).

According to aspect 1, the isolated chamber is designed with ergonomic design to accommodate the patient's head and prevent the spread of drapes and aerosols (FIG. 1 Part 4).

According to aspect 1, the hinged lids are placed on the chamber to remove the filter and create a support on the box. The chamber door with grooves of a negative angle is used to suck in drops and air (FIG. 1 Part 5).

According to aspect 1, secondary cubic air filters connected to the vent fan are used for secondary filter and absorption (FIG. 1 Part 6).

According to aspect 2, the 7-inch monitor (tablet) is used to see the patient's tooth area with the system magnification feature (FIG. 1 Part 7).

According to aspect 1, the hot and cold blast system in vent plate is to prevent the droplet and aerosol spread and to eliminate the virus with hot air. The direction of the hot wind in a negative angle causes the fluid to flow towards the fluid suction mechanism (FIG. 1 Part 8).

According to aspect 2, two angle-adjustable LED lamps have been used to keep the inside of the mouth and teeth on (FIG. 1 Part 9).

According to aspect 1, clamp connection for connecting the device to the portable base (FIG. 1 Part 10).

According to aspect 1, the device connection arm to the unit from above for baseless use of the device (FIG. 1 Part 11).

According to aspect 1, the device connection arm to the portable base with the ability to change the positioning angle of the system (FIG. 1 Part 12).

According to aspect 1, ozone is used to sterilize the final entire airflow system in the chamber and work section by the ozone generator system along with the air blast and the negative plasma ion system is intended for charged and ionization of particles and droplet (FIG. 1 Part 13).

According to aspect 2, the waterproof extra oral endoscope camera with LED is used to capture the oral environment and transfer the image to the monitor (tablet) (FIG. 1 Part 14).

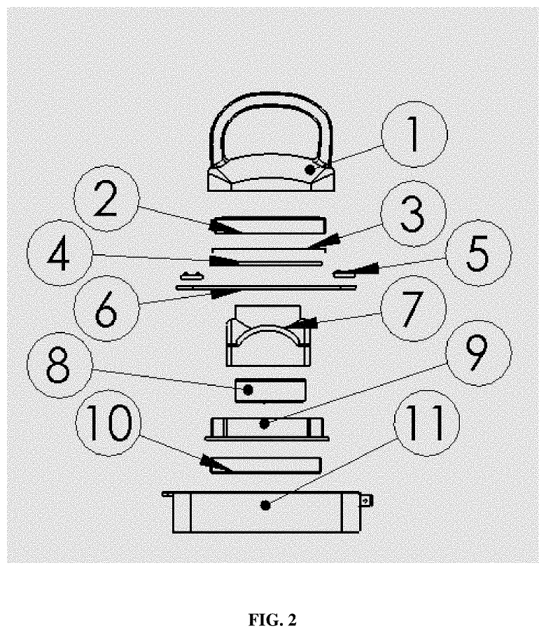

According to aspect 1 and 2, the box inside chamber is contain all electrical parts, air Ventilation system, as seen in FIG. 2.

According to aspect 1, the chamber air suction box that consist of eleven parts.

According to aspect 1, the intake air is enter from cover and nano filter entrap the virus (FIG. 1 Parts 1 and 2).

According to aspect 1, high speed turbo fan suck the fluid through manifold and transit it to final HEPA filter (FIG. 2 Part 11).

Aspect 2 of the disclosure further comprises of electronics and control parts including a camera for receiving dynamic images of the wireless controller board along with the remote and tablet as a user interface.

According to aspect 3, the input power electricity 110-220 volts is allocated to provide the energy required for the main unit.

This control unit is used to control individual modules or alone. In order to observe the principles of safe operation, the use of 12-volt elements in the device is done by switching the voltage to transmission.

According to the disclosure, there is an extra oral endoscopic camera. This waterproof camera provides a wide view of the patient's mouth to examine the maxillary teeth and transmits the projection via a communication unit cable to the tablet or monitor (FIG. 1 Part 6).

Ozone generator board and negative plasma ions are used to create ozone for sterilization at the end of the system and to create negative ionization in order to inactivate and eliminate bacteria and viruses.

Aspect 3 of the disclosure further comprises a software program application to receive and send the program performance mode according to the instructions and data due to the module selected by the dentist.

Aspect 3 of the disclosure further comprises a communication unit. This communication unit is applied for both programs in order to communicate with the host controller, it is considered as a part of the main unit, although this unit has no problem in making decisions.

Aspect 3 of the disclosure further comprises a device design system based on standard conditions and distances between the patient and the dentist is done according to the correct structure of the dentist's posture and in accordance with the four-handed dental technique and the sitting technique due to the ergonomic standard of the watch.

Aspect 3 of the disclosure is based on dentist are requirements to use the correct primer to use this device, and a corrective pattern is performed for them.

In aspect 3 of the disclosure, a wireless remote control system of this device is provided. The wireless remote control system includes on and off modules and enable or disables various parts of the device to control the performance of the device. The connection to the foot pedal to control or the connection to the Windows and in Aspect 3 or Android application operating systems to control the performance of the system can be considered.

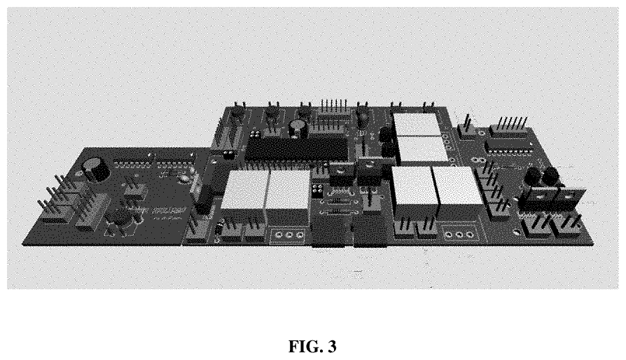

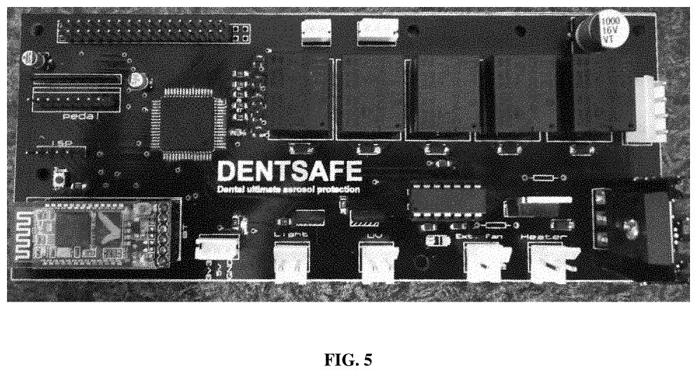

According to aspect 2 of the disclosure, all connections, wires, pipes, etc. are located in the inner wall of the chamber to control contamination. The device has electrical inputs, control boards and electrical equipment, which will be mentioned in the figures (FIG. 3 and FIG. 5). FIG. 3 is a schematic diagram showing a design PCB of Control modules. FIG. 5 is a diagram illustrating an electrical board controller for an exemplary device.

The purpose of planning to select each device module based on the dentist's work process and minimizing energy consumption is used for the ideal operating modes of the device. The use of ultraviolet light sources is intended to sterilize the device's chamber, device components and filters, even for sensitive dental instruments, this module is used individually. Six main modes (function) with the ability to adjust their power in planning were considered.

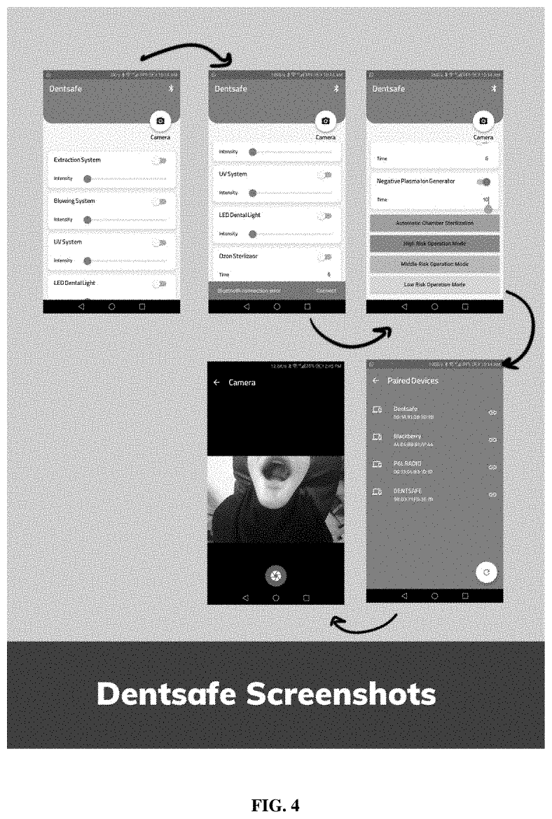

The rate of speed and suction pressure, the speed and blast pressure, and its temperature control, the amount of light intensity of the fluid and ultraviolet light, and the control of the ozone generator and the negative plasma ion device are some of the controllable modules of the system (FIG. 4).

The four automatic sterile modes of the chamber, including automatic mode adjustment for high-risk, medium and low operating modes, are important planning in the system control (FIG. 4).

According to the disclosure, a portable dental isolator apparatus for containment of contaminant discharge to prevent the transmission of infectious diseases and viruses, from the patient to the dentist is disclosed. The portal dental isolator apparatus comprises an isolation chamber guard, an air suction box containing suction turbo fan and protective dust mesh and first filter containing nano fibers and particles, a hinged lid, at plurality of LED lights, a HEPA filter, a manifold, a negative ion generator and two protective boxes containing four ultraviolet C (UVC) lamps, a display screen, an endoscopic camera, a final blast system of cold and hot air, an ozone generator and negative plasma ion, a height-adjustable base and body, a control circuit; and a wireless system and software control.

The portable dental isolator apparatus has the ability to transmit and absorb droplet and transferred aerosol from the patient to the dentist to prevent the transmission of viruses and bacteria in the fluid. The apparatus also has the ability to sterilize the surface and interior of the device capsule (chamber) and filters by the mechanism of ultraviolet (UV) lamps, and ozone also has the conditions to sterilize the external environment by using ultraviolet lamp radiation. The absorption of the aerosol fluid in the environment containing the virus by the apparatus is performing by two filtration steps.

The portable dental isolator apparatus further comprise an oral zoom system for diagnosis and treatment and taking images by an extra oral endoscopic camera. The extra oral endoscope camera may also be waterproof.

In a further embodiment, the dentist is able to transfer and move the portable dental isolator apparatus to optimally discharge negatively ionized (—OH) fluids, as well as, to correct the working position in ergonomic conditions.

The portable dental isolator apparatus further comprising an air droplet and aerosol evacuation system. This air droplet and aerosol evacuation system further comprises two stages of air filter, including a primary Nano fiber filter containing antiviral and bacterial Nano materials and activated carbon to absorb bacteria and environmental virus between the patient and the dentist (respiratory area).

The portable dental isolator apparatus further comprise a wireless system and software control application using wireless control to remotely control a computer, pedal of mobile application simultaneously or separately.

The portable dental isolator apparatus utilizes blasts of hot or cold air, along with the ionized fluid with a negative ion by eliminating the new coronavirus in the work space and the whole isolate chamber guard, to isolate and prevent infection for dentists. The hot air blast system (along with ozone blasts) with higher temperatures of 70 degrees evaporates small aerosols and disappear the coronavirus.

The placement of the surgical light, based on the standard distance between the patient's head and the dentist by examining the different ergonomic modes in dentistry, will correct the exact position of the dentist's posture.

It is noted in particular that where a range of values is provided in this specification, each value between the upper and lower limits of that range is also specifically disclosed. The upper and lower limits of these smaller ranges may independently be included or excluded in the range as well. The singular form “a”, “an”, and “the” include plural referents unless the context clearly dictates otherwise. It is intended that the specification and examples be considers as exemplary in nature and that variations that do not depart from the essence of the disclosure fall within the scope of the disclosure. Further, all of the references cited in this disclosure are each individually incorporated by reference herein their entries and as such are intended to provide an efficient way of supplementing the enabling disclosure of this disclosure, as well as, provide background detailing level of ordinary skill the art. Other features and advantages of the present disclosure will be apparent in the description of an embodiment given with reference to the drawings.

The foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure.

With respect to the above description, it is to be realized that the optimum relationships for the parts of the disclosure in regard to size, shape, form, materials, function and manner of operation, assembly and use are deemed readily apparent.

### INDUSTRIAL APPLICABILITY

One of the Industrial applications of this disclosure for investors and the dental community is that it will provide an opportunity to the new generation to provide a dental isolation device with a unique viral and bacterial epidemic and pandemic in the field of health. The urgent need for these products now and in the future plays a major role in the business market.

This disclosure could use to safe infection control treatment and could implements in personal protective equipment medical industry as well as chemical and pharmaceutical industries.

What is claimed:

1. A portable dental isolator apparatus for containment of contaminant discharge to prevent the transmission of infectious diseases and viruses, from the patient to the dentist, the apparatus comprising:
an isolation chamber guard;
an air suction box containing suction turbo fan and protective dust mesh and first filter containing nano fibers and particles;
a hinged lid;
at plurality of LED lights;
a HEPA filter;
a manifold;
a negative ion generator and two protective boxes containing four ultraviolet C (UVC) lamps;
a display screen;
an endoscopic camera;
a final blast system of cold and hot air;
an ozone generator and negative plasma ion;
a height-adjustable base and body;
a control circuit; and
a wireless system and software control.

2. The apparatus of claim 1 wherein the apparatus has the ability to transmit and absorb droplet and transferred aerosol from the patient to the dentist to prevent the transmission of viruses and bacteria in the fluid.

3. The apparatus of claim 1 wherein the apparatus has the ability to sterilize the surface and interior of the device capsule (chamber) and filters by the mechanism of ultraviolet (UV) lamps, and ozone also has the conditions to sterilize the external environment by using ultraviolet lamp radiation.

4. The apparatus of claim 3 wherein the absorption of the aerosol fluid in the environment containing the virus is performing by two filtration steps.

5. The apparatus of claim 1 further comprising an oral zoom system for diagnosis and treatment and taking images by an extra oral endoscopic camera.

6. The apparatus of claim 1, wherein the dentist is able to transfer and move the apparatus to optimally discharge negatively ionized (—OH) fluids, as well as, to correct the working position in ergonomic conditions.

7. The apparatus of claim 1 further comprising an air droplet and aerosol evacuation system, the system further comprising two stages of air filter, including a primary Nano fiber filter containing antiviral and bacterial Nano materials and activated carbon to absorb bacteria and environmental virus between the patient and the dentist (respiratory area).

8. The apparatus of claim 1 wherein the wireless system and software control uses wireless control to remotely control a computer, pedal of mobile application simultaneously or separately.

9. The apparatus of claim 1 wherein the blast of hot or cold air, along with the ionized fluid with a negative ion by eliminating the new coronavirus in the work space and the whole isolate chamber guard, is used to isolate and prevent infection for dentists.

10. The apparatus of claim 1 wherein the placement of the surgical light based on the standard distance between the patient's head and the dentist by examining the different ergonomic modes in dentistry will correct the exact position of the dentist's posture.

11. The apparatus of claim 10 wherein the hot air blast system (along with ozone) with higher temperatures of 70 degrees evaporates small aerosols and disappear the coronavirus.
